(19) United States
(12) Patent Application Publication
(10) Pub. No.: US 2007/0015686 A1
(43) Pub. Date: Jan. 18, 2007

(54) Dietary supplement for enhancing skeletal muscle mass, decreasing muscle protein degradation, downregulation of muscle catabolism pathways, and decreasing catabolism of muscle cells

(76) Inventors: Marvin A. Heuer, Mississauga (CA); Michele Molino, Mississauga (CA)

Correspondence Address: KENYON & KENYON LLP, ONE BROADWAY, NEW YORK, NY, 10004, US

(21) Appl. No.: 11/482,485

(22) Filed: Jul. 7, 2006

Related U.S. Application Data
(60) Provisional application No. 60/697,406, filed on Jul. 7, 2005.

(51) Int. Cl. A61K 38/16; A61K 31/7048; A61K 31/70; A61K 31/205; A61K 31/685; A61K 31/353

(52) U.S. Cl. 514/2; 514/23; 514/27; 514/456; 514/690; 514/78; 514/554

(57) ABSTRACT

A dietary supplement and method for enhancing skeletal muscle mass, decreasing muscle protein degradation, downregulation of muscle catabolism pathways, and decreasing catabolism of muscle cells an individual, the supplement comprising at least source of Creatine or derivatives thereof, a source of Gypenosides or Phanoside or derivatives thereof, Creatinol-O-phosphate, and a source of Epigallocatechin Gallate or derivatives thereof. The dietary supplement may further comprise N-acetyl cysteine, astaxanthin, a protein or a carbohydrate. A method of enhancing GLUT4 translocation to the plasma membrane in non-adipose cells, decreasing muscle protein degradation, downregulation of the ATP-dependent ubiquination pathway of muscle catabolism, and decreasing catabolism of muscle cells through reducing the activation of NF-κμ is also provided.

### RELATED APPLICATIONS

This application is related to and claims benefit of priority to Applicant's co-pending U.S. Provisional Patent Application Ser. No. 60/697,406, entitled “Nutritional composition for enhancing skeletal muscle mass, increasing muscle fatigue resistance and recovery, augmenting muscle glycogen deposition rate, preventing skeletal muscle protein catabolism, and/or reducing muscle soreness and inflammation,” filed Jul. 7, 2005, the disclosure of which is hereby fully incorporated by reference.

### FIELD OF THE INVENTION

The present invention relates to a dietary supplement, and more particularly to a dietary supplement for enhancing GLUT4 protein translocation to the plasma membrane in non-adipose cells, decreasing muscle protein degradation, downregulation of the ATP-dependent ubiquination pathway of muscle catabolism, and decreasing catabolism of muscle cells through reducing the activation of NF-κμ.

### SUMMARY OF THE INVENTION

The present invention relates to a dietary supplement for enhancing GLUT4 protein translocation to the plasma membrane in non-adipose cells, decreasing muscle protein degradation, downregulation of the ATP-dependent ubiquination pathway of muscle catabolism, and decreasing catabolism of muscle cells through reducing the activation of NF-κβ. More specifically, the present invention relates to a novel dietary supplement comprising at least a source of Creatine or derivatives thereof, a source of Gypenosides or Phanosides, Creatinol-O-phosphate, and a source of Epigallocatechin Gallate or derivatives thereof. Additionally, the present invention may comprise N-acetyl cysteine, and astaxanthin. The present invention may also comprise a protein or a source of protein and amino acids as well as a carbohydrate or a source of carbohydrates or sugars. Furthermore, a method for achieving the same by way of administration of the composition is presented.

For example, the present invention is related to a novel diet supplement for decreasing muscle catabolism and increasing muscle size and strength. Furthermore, the present invention provides a method for enhancing GLUT4 protein translocation to the plasma membrane of non-adipose cells. The diet supplement is particularly advantageous for individuals, e.g. a human or an animal seeking to increase muscle size and/or muscle strength. The diet supplement of the present invention comprises a source of catechins, such as epigallocatechin gallate, epicatechin gallate, epicatechin and/or tannic acid, as well as further comprising a source of Gypenosides. Furthermore, the present invention may comprise a source of Proteins or amino acids or derivatives thereof, a source Carbohydrates or derivatives thereof, N-acetyl cysteine, Astaxanthin, Creatine, and/or Creatine-O-Phosphate. Furthermore, by way of consumption of the diet supplement, the present invention provides a method of decreasing muscle catabolism and increasing muscle size and strength and enhancing GLUT4 protein translocation to the plasma membrane of non-adipose cells.

### DETAILED DESCRIPTION OF THE INVENTION

The present invention, according to various embodiments thereof, is directed to a dietary supplement for enhancing GLUT4 protein translocation to the plasma membrane in non-adipose cells, decreasing muscle protein degradation, downregulation of the ATP-dependent ubiquination pathway of muscle catabolism, and decreasing catabolism of muscle cells through reducing the activation of NF-κβ. The dietary supplement may comprise one or more of high to moderate-glycemic index carbohydrates, dammarane saponins from Gynostemma pentaphyllum, ester-bond containing polyphenols, and creatine and related guanidine compounds. According to various embodiments of the present invention, the dietary supplement may additionally comprise Creatinol-O-phosphate as a source of guanidino compounds. The dietary supplement may also further comprise the antioxidant N-acetyl cysteine (NAC) and the carotenoid, astaxanthin. Furthermore, the dietary supplement may include one or more of a number of branched-chain amino acids and essential amino acids.

Definitions

As used herein, “a Carbohydrate” refers to at least a source of carbohydrates such as, but not limited to, a monosaccharide, disaccharide, polysaccharide or derivatives thereof.

As used herein, “a Protein” refers to at least a source of protein or amino acids.

As used herein, “Branched-chain amino acid” refers to at least a source of one of the amino acids leucine, isoleucine or valine.

As used herein, “Essential amino acid” refers to at least a source of one of the amino acids: tryptophan, lysine, methionine, phenylalanine, threonine, valine, leucine, isoleucine and histidine.

As used herein, “Creatine” refers to the chemical N-methyl-N-guanyl Glycine, (CAS Registry No. 57-00-1), also known as, (alpha-methyl guanido) acetic acid, N-(aminoiminomethyl)-N-glycine, Methylglycocyamine, Methylguanidoacetic Acid, or N-Methyl-N-guanylglycine, whose chemical structure is shown below. Additionally, as used herein, “Creatine” also includes derivatives of Creatine such as esters, and amides, and salts, as well as other derivatives, including derivatives that become active upon metabolism. Furthermore, Creatinol (CAS Registry No. 6903-79-3), also known as Creatine-O-Phosphate, N-methyl-N-(beta-hydroxyethyl)guanidine O-phosphate, Aplodan, or 2-(carbamimidoyl-methyl-amino)ethoxyphosphonic acid, is henceforth in this disclosure considered to be a creatine derivative.

Furthermore, for the purposes of this disclosure, examples of ester-bond containing polyphenols may include, but are not limited to, epigallocatechin gallate (EGCG), epigallocatechin (EGC), epicatechin gallate (ECG), epicatechin (EC), and gallocatechin gallate (GCG), or hydrolysable tannins.

Muscle growth may be optimized by combining exercise and appropriate nutritional strategies. The effects of combined exercise and nutritional strategies are integrated at the level of one central regulatory protein, mTOR (mammalian target of rapamycin) (Dann S G, Thomas G. The amino acid sensitive TOR pathway from yeast to mammals. FEBS Left. 2006 May 22; 580(12):2821-9.; Deldicque L, Theisen D, Francaux M. Regulation of mTOR by amino acids and resistance exercise in skeletal muscle. Eur J Appl Physiol. 2005 May; 94(1-2):1-10). mTOR is a complex protein containing several regulatory sites as well as sites for interaction with multiple other proteins which acts by integrating signals of the energetic status of the cell and environmental stimuli to control protein synthesis, protein breakdown and, therefore, cell growth (Hay N, Sonenberg N. Upstream and downstream of mTOR. Genes Dev. 2004 Aug. 15; 18(16):1926-45). The mTOR kinase controls the translation machinery, in response to amino acids and growth factors, such as insulin and insulin-like growth factor 1 (IGF-1), via the activation of p70 ribosomal 86 kinase (p70S6K) and the inhibition of eIF-4E binding protein (4E-BP1). Furthermore, the mTOR protein is a member of the PI3K pathway and functions through the involvement of the Akt kinase, an upstream regulator of mTOR (Asnaghi L, Bruno P, Priulla M, Nicolin A. mTOR: a protein kinase switching between life and death. Pharmacol Res. 2004 December; 50(6):545-9). For example, e.g., interaction of insulin with receptors leads to the cell membrane recruitment and stimulation of PI3K and production of the messenger PIP3 (Chung J, Grammer T C, Lemon K P, Kazlauskas A, Blenis J. PDGF- and insulin-dependent pp70S6k activation mediated by phosphatidylinositol-3-OH kinase. Nature. 1994 Jul. 7; 370(6484):71-5) which in turn binds to pro-survival kinase PKB/AKT (Dufner A, Andjelkovic M, Burgering B M, Hemmings B A, Thomas G. Protein kinase B localization and activation differentially affect S6 kinase 1 activity and eukaryotic translation initiation factor 4E-binding protein 1 phosphorylation. Mol Cell Biol. 1999 June; 19(6):4525-34), leading to the activation of mTOR (Long X, Lin Y, Ortiz-Vega S, Yonezawa K, Avruch J. Rheb binds and regulates the mTOR kinase. Curr Biol. 2005 Apr. 26; 15(8):702-13). Activated mTOR then phosphorylates 4E-BP1 causing it to dissociate from eIF-4E (Brunn G J, Hudson C C, Sekulic A, Williams J M, Hosoi H, Houghton P J, Lawrence J C Jr, Abraham R T. Phosphorylation of the translational repressor PHAS-I by the mammalian target of rapamycin. Science. 1997 Jul. 4; 277(5322):99-101). Once dissociated, eIF-4E is able to participate in translation. Moreover, several substrates, related to protein synthesis and cell growth of the mTOR effector kinase p70S6K have been identified. (Dann S G, Thomas G. The amino acid sensitive TOR pathway from yeast to mammals. FEBS Lett. 2006 May 22; 580(12):2821-9).

The P13K/Akt/mTOR pathway, has been characterized as being critical for net muscle gain and/or hypertrophy. It is also necessary that it be active in order for IGF-1-mediated transcriptional changes to occur and inversely regulate atrophy-induced genes. IGF-1 stimulates essential transcription from RNA polymerase I (James M J, Zomerdijk J C. Phosphatidylinositol 3-kinase and mTOR signaling pathways regulate RNA polymerase I transcription in response to IGF-1 and nutrients. J Biol Chem. 2004 Mar. 5; 279(10):8911-8). This stimulation is dependent on PI3K and is mediated via mTOR. IGF-1 has also been shown to inversely regulate a subset of genes involved in atrophy, thereby reducing atrophy via its involvment (Latres E, Amini A R, Amini A A, Griffiths J, Martin F J, Wei Y, Lin H C, Yancopoulos G D, Glass D J. Insulin-like growth factor-1 (IGF-1) inversely regulates atrophy-induced genes via the phosphatidylinositol 3-kinase/Akt/mammalian target of rapamycin (PI3K/Akt/mTOR) pathway. J Biol Chem. 2005 Jan. 28; 280(4):2737-44).

The expression of the MAFbx, e.g., atorpin-1, a ubiquitin-ligase, a muscle atrophy F-box gene, is inhibited by IGF-1 as well as insulin (Sacheck J M, Ohtsuka A, McLary S C, Goldberg A L. IGF-I stimulates muscle growth by suppressing protein breakdown and expression of atrophy-related ubiquitin ligases, atrogin-1 and MuRF1. Am J Physiol Endocrinol Metab. 2004 October; 287(4):E591-601) by way of inhibiting FOXO transcription factors (Stitt T N, Drujan D, Clarke B A, Panaro F, Timofeyva Y, Kline W O, Gonzalez M, Yancopoulos G D, Glass D J. The IGF-1/PI3K/Akt pathway prevents expression of muscle atrophy-induced ubiquitin ligases by inhibiting FOXO transcription factors. Mol Cell. 2004 May 7; 14(3):395-403) which control the expression of MAFbx. This further strengthens the need for IGF-1 in shifting the anabolism/catabolism balance in order for hypertrophy to occur.

Upstream signaling, by nutrients, of mTOR, particularly amino acids, has been shown to modulate different downstream signaling branches through interaction with various intracellular and/or membrane-bound extracellular amino acid sensors (Dann S G, Thomas G. The amino acid sensitive TOR pathway from yeast to mammals. FEBS Lett. 2006 May 22; 580(12):2821-9). Moreover, exercise and amino acid modulation of mTOR use different signaling pathways upstream of mTOR, for example, e.g., exercise seems to recruit partially the same pathway as insulin, whereas amino acids could act more directly on mTOR (Deldicque L, Theisen D, Francaux M. Regulation of mTOR by amino acids and resistance exercise in skeletal muscle. Eur J Appl Physiol. 2005 May; 94(1-2):1-10). The 5'AMP-activated protein kinase (AMPK) is regulated by changes in ATP levels. When ATP levels drop, as they do rapidly during resistance exercise, AMPK is activated. This activation of AMPK decreases mTOR activity in a manner similar to the effect of glucose deprivation (Kimura N, Tokunaga C, Dalal S, Richardson C, Yoshino K, Hara K, Kemp B E, Witters L A, Mimura O, Yonezawa K. A possible linkage between AMP-activated protein kinase (AMPK) and mammalian target of rapamycin (mTOR) signalling pathway. Genes Cells. 2003 January; 8(1):65-79). AMPK plays an important role in relaying energy availability and nutrient/hormonal signals to control appetite and body weight (Minokoshi Y, Alquier T, Furukawa N, Kim Y B, Lee A, Xue B, Mu J, Foufelle F, Ferre P, Birnbaum M J, Stuck B J, Kahn B B. AMP-kinase regulates food intake by responding to hormonal and nutrient signals in the hypothalamus. Nature. 2004 Apr. 1; 428(6982):569-74). During recovery immediately following exercise, the inhibition of mTOR by AMPK is suppressed, and its activation is maximized by the presence of amino acids and allowed by the permissive role of insulin (Deldicque L, Theisen D, Francaux M. Regulation of mTOR by amino acids and resistance exercise in skeletal muscle. Eur J Appl Physiol. 2005 May; 94(1-2):1-10; Bolster DR, Kubica N, Crozier S J, Williamson DL, Farrell P A, Kimball S R, Jefferson L S. Immediate response of mammalian target of rapamycin (mTOR)-mediated signalling following acute resistance exercise in rat skeletal muscle. J Physiol. 2003 Nov. 15; 553(Pt 1):213-20).

Resistance exercise disturbs skeletal muscle homeostasis leading to activation of catabolic (breakdown) and anabolic (synthesis) processes within the muscle cell. Generally, resistance exercise stimulates muscle protein synthesis more than breakdown such that the net muscle protein balance (e.g., synthesis minus breakdown) is in favor of increasing muscle (Biolo G, Maggi S P, Williams B D, Tipton K D, Wolfe R R. Increased rates of muscle protein turnover and amino acid transport after resistance exercise in humans. Am J Physiol. 1995 March; 268(3 Pt 1):E514-20). However, exercise-induced increases in protein synthesis may not be stimulated until several hours following exercise (Hernandez J M, Fedele M J, Farrell P A. Time course evaluation of protein synthesis and glucose uptake after acute resistance exercise in rats. J Appl Physiol. 2000 March; 88(3):1142-9), albeit, in the absence of adequate nutritional intake in the period after exercise, the balance shifts in favor of protein catabolism (Biolo G, Maggi S P, Williams B D, Tipton K D, Wolfe R R. increased rates of muscle protein turnover and amino acid transport after resistance exercise in humans. Am J Physiol. 1995 March; 268(3 Pt 1):E514-20; Biolo G, Tipton K D, Klein S, Wolfe R R. An abundant supply of amino acids enhances the metabolic effect of exercise on muscle protein. Am J Physiol. 1997 July; 273(1 Pt 1):E122-9). Consequently, during the time that resistance exercise is being performed and for a time period following exercise, there may be a net loss of muscle protein because protein synthesis is either decreased (Bylund-Fellenius A C, Ojamaa K M, Flaim K E, Li J B, Wassner S J, Jefferson L S. Protein synthesis versus energy state in contracting muscles of perfused rat hindlimb. Am J Physiol. 1984 April; 246(4 Pt 1):E297-305) or remains unchanged (Carraro F, Stuart C A, Hartl W H, Rosenblatt J. Wolfe R R. Effect of exercise and recovery on muscle protein synthesis in human subjects. Am J Physiol. 1990 October; 259(4 Pt 1):E470-6), whereas protein breakdown is generally considered to be elevated (Rennie M J, Edwards R H, Krywawych S, Davies C T, Halliday D, Waterlow J C, Millward D J. Effect of exercise on protein turnover in man. Clin Sci (Lond). 1981 November; 61(5):627-39). It would be advantageous, for that reason, to limit the activity of proteolytic mechanisms during the exercise bout.

Carbohydrate ingestion stimulates the secretion of insulin which in turn facilitates the uptake of glucose into skeletal muscles and the liver and promotes its storage as glycogen and triglycerides. Concomitant with this, insulin inhibits the release and synthesis of glucose (Khan A H, Pessin J E. Insulin regulation of glucose uptake: a complex interplay of intracellular signalling pathways. Diabetologia. 2002 November; 45(11):1475-83). Moreover, insulin also has an important role in protein metabolism—the inhibition of the breakdown of protein or proteolysis (Volpi E and Wolfe B. Insulin and Protein Metabolism. In: Handbook of Physiology, L. Jefferson and A. Cherrington editors. New York: Oxford, 2001, p. 735-757; Boirie Y, Gachon P, Cordat N, Ritz P, Beaufrere B. Differential insulin sensitivities of glucose, amino acid, and albumin metabolism in elderly men and women. J. Clin Endocrinol Metab. 2001 February; 86(2):638-44). Furthermore, in the presence of a sufficient concentration of amino acids, insulin will promote the uptake of amino acids into muscle and stimulate protein synthesis (Tessari P, Inchiostro S, Biolo G, Trevisan R, Fantin G, Marescotti M C, lori E, Tiengo A, Crepaldi G. Differential effects of hyperinsulinemia and hyperaminoacidemia on leucine-carbon metabolism in vivo. Evidence for distinct mechanisms in regulation of net amino acid deposition. J Clin Invest. 1987 April; 79(4):1062-9; Biolo G, Declan Fleming R Y, Wolfe R R. Physiologic hyperinsulinemia stimulates protein synthesis and enhances transport of selected amino acids in human skeletal muscle. J Clin Invest. 1995 February; 95(2):811-9), particularly following exercise (Biolo G, Williams B D, Fleming R Y, Wolfe R R. Insulin action on muscle protein kinetics and amino acid transport during recovery after resistance exercise. Diabetes. 1999 May; 48(5):949-57). When carbohydrates and amino acids are combined, an additive net effect on protein synthesis is observed (Miller S L, Tipton K D, Chinkes D L, Wolf S E, Wolfe R R. Independent and combined effects of amino acids and glucose after resistance exercise. Med Sci Sports Exerc. 2003 March; 35(3):449-55). Studies have shown that the ingestion of carbohydrates with amino acids can ameliorate muscle atrophy due to prolonged inactivity or bed-rest (Paddon-Jones D, Sheffield-Moore M, Urban R J, Sanford A P, Aarsland A, Wolfe R R, Ferrando A A. Essential amino acid and carbohydrate supplementation ameliorates muscle protein loss in humans during 28 days bedrest. J Clin Endocrinol Metab. 2004 September; 89(9):4351-8).

The work by Tipton and colleagues (Tipton K D, Rasmussen B B, Miller S L, Wolf S E, Owens-Stovall S K, Petrini B E, Wolfe R R. Timing of amino acid-carbohydrate ingestion alters anabolic response of muscle to resistance exercise. Am J Physiol Endocrinol Metab. 2001 August; 281(2):E197-206) has shown that the ingestion of an amino acid-carbohydrate supplement in the immediate pre-workout period, by promoting hyperinsulinemia while an intense resistance exercise session is being performed, is capable of limiting muscle protein breakdown. This may occur since the carbohydrates are utilized for energy production instead of muscular or exogenous amino acids, which, in the absence of adequate amounts of blood sugars, would be alternatively spent as a source of metabolic fuel, thereby promoting muscle protein breakdown and/or impairment of new protein synthesis.

Glucose transporter 4 (GLUT4) is responsible for insulin-dependent glucose uptake into skeletal muscle. In the basal state, GLUT4 is predominantly found within intracellular vesicles. Insulin stimulation initiates a signaling cascade that results in these intracellular vesicles containing GLUT4 to translocate and fuse to the plasma membrane. The activation of Akt by insulin is involved in this translocation of GLUT4. In the insulin-stimulated state in muscle cells, more than 90% of the GLUT4 is located at the plasma membrane (Wang W, Hansen P A, Marshall B A, Holloszy J O, Mueckler M. Insulin unmasks a COOH-terminal Glut4 epitope and increases glucose transport across T-tubules in skeletal muscle. J Cell Biol. 1996 October; 135(2):415-30; Mueckler M. Insulin resistance and the disruption of Glut4 trafficking in skeletal muscle. J Clin Invest. 2001 May; 107(10):1211-3). GLUT4 docking and fusion to skeletal muscle plasma membrane is regulated by the activity of soluble N-ethylmaleimide-senstive fusion protein attachment receptors (SNAREs), a family of membrane proteins that target specificity in the vacuolar system and control fusion reactions by forming fusion-competent structures composed of SNAREs from each of the fusing membranes. Particularly, the insulin-stimulated plasma membrane docking and fusion of GLUT4 vesicles appears to require specific interactions between the plasma membrane t-SNARE proteins, Syntaxin 4 and SNAP23, with the GLUT4 vesicle v-SNARE protein, VAMP2 (Cheatham B, Volchuk A, Kahn C R, Wang L, Rhodes C J, Klip A. Insulin-stimulated translocation of GLUT4 glucose transporters requires SNARE-complex proteins. Proc Natl Acad Sci USA. 1996 Dec. 24; 93(26):15169-73; Volchuk A, Wang Q, Ewart H S, Liu Z, He L, Bennett M K, Klip A. Syntaxin 4 in 3T3-L1 adipocytes: regulation by insulin and participation in insulin-dependent glucose transport. Mol Biol Cell. 1996 July; 7(7):1075-82; Martin L B, Shewan A, Millar C A, Gould G W, James D E. Vesicle-associated membrane protein 2 plays a specific role in the insulin-dependent trafficking of the facilitative glucose transporter GLUT4 in 3T3-L1 adipocytes. J Biol Chem. 1998 Jan. 16; 273(3):1444-52; Kawanishi M, Tamori Y, Okazawa H, Araki S, Shinoda H, Kasuga M. Role of SNAP23 in insulin-induced translocation of GLUT4 in 3T3-L1 adipocytes. Mediation of complex formation between syntaxin4 and VAMP2. J Biol Chem. 2000 Mar. 17; 275(11):8240-7).

Experiments have demonstrated that selective blocking of Syntaxin 4 activity inhibits insulin-stimulated GLUT4 translocation at the skeletal muscle plasma membrane and causes insulin insensitivity (Volchuk A, Wang Q, Ewart H S, Liu Z, He L, Bennett M K, Klip A. Syntaxin 4 in 3T3-L1 adipocytes: regulation by insulin and participation in insulin-dependent glucose transport. Mol Biol Cell. 1996 July; 7(7):1075-82; Martin L B, Shewan A, Millar C A, Gould G W, James D E. Vesicle-associated membrane protein 2 plays a specific role in the insulin-dependent trafficking of the facilitative glucose transporter GLUT4 in 3T3-L1 adipocytes. J Biol Chem. 1998 Jan. 16; 273(3):1444-52; Kawanishi M, Tamori Y, Okazawa H, Araki S, Shinoda H, Kasuga M. Role of SNAP23 in insulin-induced translocation of GLUT4 in 3T3-L1 adipocytes. Mediation of complex formation between syntaxin4 and VAMP2. J Biol Chem. 2000 Mar. 17; 275(11):8240-7; Yang C, Coker K J, Kim J K, Mora S, Thurmond D C, Davis A C, Yang B, Williamson R A, Shulman G I, Pessin J E. Syntaxin 4 heterozygous knockout mice develop muscle insulin resistance. J Clin Invest. 2001 May; 107(10):1311-8), whereas insulin-stimulated GLUT4 translocation seems not to be impaired in adipocytes, suggesting the existence of other mechanisms for GLUT4 translocation in adipose tissue (Yang C, Coker K J, Kim J K, Mora S, Thurmond D C, Davis A C, Yang B, Williamson R A, Shulman G I, Pessin J E. Syntaxin 4 heterozygous knockout mice develop muscle insulin resistance. J Clin Invest. 2001 May; 107(10):1311-8). Recent evidence has shown that proteins of the Syntaxin family (e.g., Syntaxin1) can be targeted by specific ubiquitin-protein ligases to facilitate their ubiquitination and proteasome-dependent degradation (Chin L S, Vavalle J P, Li L. Staring, a novel E3 ubiquitin-protein ligase that targets syntaxin 1 for degradation. J Biol Chem. 2002 Sep. 20; 277(38):35071-9). This effect may produce reduced glucose uptake in skeletal muscle but enhanced glucose uptake in adipose tissue, as demonstrated by the circumstance that GLUT4 expression in adipocytes is repressed by proteasome inhibition (Cooke D W, Patel Y M. GLUT4 expression in 3T3-L1 adipocytes is repressed by proteasome inhibition, but not by inhibition of calpains. Mol Cell Endocrinol. 2005 Mar. 31; 232(1-2):37-45).

Further to limiting the general activity of proteolytic mechanisms responsible for muscle catabolism during and immediately following an exercise bout, it would be advantageous to limit the ubiquitination and proteasome-dependent degradation of Syntaxins in order to prolong the time of permanence of the glucose transporter at the plasma membrane of skeletal muscle fibers, therefore favoring the maximization of glucose influx in this tissue.

Sustained plasma insulin levels would be able to limit muscle protein catabolism by interfering with the signaling pathways of the ATP-dependent ubiquitin/proteasome proteolytic complex, e.g., the macromolecular cytosolic multi-catalytic complex responsible for protein degradation and turnover, and the major intracellular target of the antiproteolytic action of insulin (Hamel F G, Bennett R G, Harmon K S, Duckworth W C. Insulin inhibition of proteasome activity in intact cells. Biochem Biophys Res Commun. 1997 May 29; 234(3):671-4; Duckworth W C, Bennett R G, Hamel F G. Insulin acts intracellularly on proteasomes through insulin-degrading enzyme. Biochem Biophys Res Commun. 1998 Mar. 17; 244(2):390-4; Bennett R G, Hamel F G, Duckworth W C. Insulin inhibits the ubiquitin-dependent degrading activity of the 26S proteasome. Endocrinology. 2000 July; 141(7):2508-17; Bennett R G, Fawcett J, Kruer M C, Duckworth W C, Hamel F G. Insulin inhibition of the proteasome is dependent on degradation of insulin by insulin-degrading enzyme. J Endocrinol. 2003 June; 177(3):399-405). The proteolytic activity of the ubiquitin/proteasome complex can be activated by: excessive cytokine and glucocorticoids release (e.g., during the occurrence of stress, overtraining conditions, injury, trauma, infection, inflammation, fasting etc.), ageing, protracted critical illness, and wasting syndromes (like, for instance, cancer, HIV and chronic obstructive pulmonary disease—COPD) (Glickman M H, Ciechanover A. The ubiquitin-proteasome proteolytic pathway: destruction for the sake of construction. Physiol Rev. 2002 April; 82(2):373-428; Attaix D, Combaret L, Pouch M N, Taillandier D. Regulation of proteolysis. Curr Opin Clin Nutr Metab Care. 2001 January; 4(1):45-9; Wilkinson K D. Roles of ubiquitinylation in proteolysis and cellular regulation. Annu Rev Nutr. 1995; 15:161-89; Smith L L. Cytokine hypothesis of overtraining: a physiological adaptation to excessive stress? Med Sci Sports Exerc. 2000 Febraury; 32(2):317-31; Jackman R W, Kandarian S C. The molecular basis of skeletal muscle atrophy. Am J Physiol Cell Physiol. 2004 October; 287(4):C834-43; Mansoor O, Beaufrere B, Boirie Y, Ralliere C, Taillandier D, Aurousseau E, Schoeffler P, Arnal M, Attaix D. Increased mRNA levels for components of the lysosomal, Ca2+-activated, and ATP-ubiquitin-dependent proteolytic pathways in skeletal muscle from head trauma patients. Proc Natl Acad Sci U S A. 1996 Apr. 2; 93(7):2714-8).

The ability of insulin to inhibit the proteolytic activity of the ubiquitin/proteasome complex is wide-ranging. First, insulin can decrease the catalytic activity of the proteasome by inhibiting its peptide-degrading action (Duckworth W C, Bennett R G, Hamel F G. A direct inhibitory effect of insulin on a cytosolic proteolytic complex containing insulin-degrading enzyme and multicatalytic proteinase. J Biol Chem. 1994 Oct. 7; 269(40):24575-80). Second, insulin has been shown to interfere with and downregulate the ATP-dependent ubiquitin (Ub) pathway at the level of Ub conjugation (Roberts R G, Redfern C P, Goodship T H. Effect of insulin upon protein degradation in cultured human myocytes. Eur J Clin Invest. 2003 October; 33(10):861-7; Price S R, Bailey J L, Wang X, Jurkovitz C, England B K, Ding X, Phillips L S, Mitch W E. Muscle wasting in insulinopenic rats results from activation of the ATP-dependent, ubiquitin-proteasome proteolytic pathway by a mechanism including gene transcription. J Clin Invest. 1996 Oct. 15; 98(8):1703-8; Mitch W E, Bailey J L, Wang X, Jurkovitz C, Newby D, Price S R. Evaluation of signals activating ubiquitin-proteasome proteolysis in a model of muscle wasting. Am J Physiol. 1999 May; 276(5 Pt 1):C1132-8) if, for example, the biochemical mechanism that allows the marking of proteins destined for degradation in order that they can be recognized and degraded by the 26S proteasome (Lecker S H, Solomon V, Mitch W E, Goldberg A L. Muscle protein breakdown and the critical role of the ubiquitin-proteasome pathway in normal and disease states. J Nutr. 1999 January; 129(1S Suppl):227S-237S). This anti-catabolic action of insulin is particularly important when muscle protein degradation is derived as a result of the effects of glucocorticoids for example, e.g., during fasting, immobilization, and in conditions of extreme metabolic stress (Lecker S H, Solomon V, Mitch W E, Goldberg A L. Muscle protein breakdown and the critical role of the ubiquitin-proteasome pathway in normal and disease states. J Nutr. 1999 January; 129(1S Suppl):227S-237S; Wing S S, Haas A L, Goldberg A L. Increase in ubiquitin-protein conjugates concomitant with the increase in proteolysis in rat skeletal muscle during starvation and atrophy denervation. Biochem J. 1995 May 1; 307 (Pt 3):639-45). Third, as aformentioned insulin and/or IGF-I reduce the expression of MAFbx, a muscle-specific Ub-ligase required for muscle atrophy. MAFbx expression is induced several folds during fasting and in many wasting disease states, as shown by experimental evidence (Gomes M D, Lecker S H, Jagoe R T, Navon A, Goldberg A L. Atrogin-1, a muscle-specific F-box protein highly expressed during muscle atrophy. Proc Natl Acad Sci USA. 2001 Dec. 4; 98(25):14440-5; Sacheck J M, Ohtsuka A, McLary S C, Goldberg A L. IGF-I stimulates muscle growth by suppressing protein breakdown and expression of atrophy-related ubiquitin ligases, atrogin-1 and MuRF1. Am J Physiol Endocrinol Metab. 2004 October; 287(4):E591-601). This multifaceted action of insulin, in conjunction with the downregulating action of amino acids on essential components of the Ub system (Hamel F G, Fawcett J, Bennett R G, Duckworth W C. Control of proteolysis: hormones, nutrients, and the changing role of the proteasome. Curr Opin Clin Nutr Metab Care. 2004 May; 7(3):255-8) ultimately reduces the deleterious effects of excessive ATP-dependent Ub/proteasome complexing on skeletal muscle mass and myofibrillar protein.

Experimental studies have demonstrated that ester bond-containing polyphenols, such as EGCG and ECG catechins, at concentrations found in the serum of green tea drinkers, and hydrolysable tannins, for example, tannic acid (TA) or complex tannins, are potent specific inhibitors of the chymotrypsin-like activity of the previously mentioned proteasome complex both in vitro and in vivo (Nam S, Smith D M, Dou Q P. Ester bond-containing tea polyphenols potently inhibit proteasome activity in vitro and in vivo. J Biol Chem. 2001 Apr. 20; 276(16):13322-30; Kazi A, Urbizu D A, Kuhn D J, Acebo A L, Jackson E R, Greenfelder G P, Kumar N B, Dou Q P. A natural musaceas plant extract inhibits proteasome activity and induces apoptosis selectively in human tumor and transformed, but not normal and non-transformed, cells. Int J Mol Med. 2003 December; 12(6):879-87; Nam S, Smith D M, Dou Q P. Tannic acid potently inhibits tumor cell proteasome activity, increases p27 and Bax expression, and induces G1 arrest and apoptosis. Cancer Epidemiol Biomarkers Prev. 2001 October; 10(10):1083-8; Kuhn D J, Burns A C, Kazi A, Dou Q P. Direct inhibition of the ubiquitin-proteasome pathway by ester bond-containing green tea polyphenols is associated with increased expression of sterol regulatory element-binding protein 2 and LDL receptor. Biochim Biophys Acta. 2004 Jun. 1; 1682(1-3):1-10). The inhibition of said proteasome by ester bond-containing catechins and TA results in an accumulation of the inhibitor protein Iκβ-α, which, in turn, inhibits transcription factor nuclear factor-κβ (NF-κβ) translocation to the nucleus, thereby preventing its transcriptional activity and the accelerated activation of muscle protein degradation (Langen R C, Schols A M, Kelders M C, Wouters E F, Janssen-Heininger Y M. Inflammatory cytokines inhibit myogenic differentiation through activation of nuclear factor-kappaB. FASEB J. 2001 May; 15(7):1169-80; Karin M. The beginning of the end: IkappaB kinase (IKK) and NF-kappaB activation. J Biol Chem. 1999 Sep. 24; 274(39):27339-42).

In addition, recent evidence suggests that plant extracts rich in EGCG and ECG have the ability to improve post-prandial glucose metabolism in healthy humans and animals, as well, they have been shown to produce an anti-hyperglycemic effect in animal models of diabetes (Tsuneki H, Ishizuka M, Terasawa M, Wu J B, Sasaoka T, Kimura I. Effect of green tea on blood glucose levels and serum proteomic patterns in diabetic (db/db) mice and on glucose metabolism in healthy humans. BMC Pharmacol. 2004 Aug. 26; 4:18; Waltner-Law M E, Wang X L, Law B K, Hall R K, Nawano M, Granner D K. Epigallocatechin gallate, a constituent of green tea, represses hepatic glucose production. J Biol Chem. 2002 Sep. 20; 277(38):34933-40; Ashida H, Furuyashiki T, Nagayasu H, Bessho H, Sakakibara H, Hashimoto T, Kanazawa K. Anti-obesity actions of green tea: possible involvements in modulation of the glucose uptake system and suppression of the adipogenesis-related transcription factors. Biofactors. 2004; 22(1-4):135-40). EGCG and ECG derivatives have been shown to enhance insulin metabolism by selective stimulation of GLUT4 translocation to skeletal muscle plasma membrane, selective enhancement of glycogenesis in skeletal muscles, simultaneous downregulation of GLUT4 translocation to adipose cells membrane, and reduced expression/activity of adipogenesis-related transcription factors (therefore preventing the utilization of glucose for lipogenic purposes) (Ashida H, Furuyashiki T, Nagayasu H, Bessho H, Sakakibara H, Hashimoto T, Kanazawa K. Anti-obesity actions of green tea: possible involvements in modulation of the glucose uptake system and suppression of the adipogenesis-related transcription factors. Biofactors. 2004; 22(1-4): 135-40).

During inflammation, sepsis, infection, excessive physical stress, chronic illness and in aging, plasma and tissue concentrations of essential inflammatory cytokines, principally those of the tumor necrosis factor-α (TNF-α) and interleukin-1β (IL-1μ) superfamilies, increase dramatically (Norman M U, Lister K J, Yang Y H, Issekutz A, Hickey M J. TNF regulates leukocyte-endothelial cell interactions and microvascular dysfunction during immune complex-mediated inflammation. Br J Pharmacol. 2005 January; 144(2):265-74; Nemet D, Oh Y, Kim H S, Hill M, Cooper D M. Effect of intense exercise on inflammatory cytokines and growth mediators in adolescent boys. Pediatrics. 2002 October; 110(4):681-9). Excessive TNF-α concentration in plasma and tissues initiates a deleterious cycle of catabolic and degradative events mediated via activation of the transcription factor NF-κβ. This circumstance ultimately leads to hypercortisolism, decreased levels of somatotropic hormones, for example, Growth Hormone and IGF-I, disturbed protein balance, loss of muscle protein stores, systemic inflammation and compromised immune functions (Smith L L. Cytokine hypothesis of overtraining: a physiological adaptation to excessive stress? Med Sci Sports Exerc. 2000 February; 32(2):317-31; Steinacker J M, Lormes W, Reissnecker S, Liu Y. New aspects of the hormone and cytokine response to training. Eur J Appl Physiol. 2004 April; 91 (4):382-91).

Numerous lines of evidence support the role of TNF-α as a prominent mediator of accelerated skeletal muscle protein degradation (cachexia) and declined insulin sensitivity as seen in severe inflammatory conditions, chronic wasting syndromes, aging, diabetes and obesity (Steinacker J M, Lormes W, Reissnecker S, Liu Y. New aspects of the hormone and cytokine response to training. Eur J Appl Physiol. 2004 April; 91(4):382-91; Lang C H, Hong-Brown L, Frost R A. Cytokine inhibition of JAK-STAT signaling: a new mechanism of growth hormone resistance. Pediatr Nephrol. 2005 March; 20(3):306-12; Kirwan J P, Krishnan R K, Weaver J A, Del Aguila L F, Evans W J. Human aging is associated with altered TNF-alpha production during hyperglycemia and hyperinsulinemia. Am J Physiol Endocrinol Metab. 2001 December; 281(6):E1137-43; Hotamisligil G S. The role of TNFalpha and TNF receptors in obesity and insulin resistance. J Intern Med. 1999 June; 245(6):621-5).

In overtraining, excessive muscle production of pro-inflammatory cytokines for example, e.g. IL-1β and TNF-α, induces a myopathy-like state characterized by exercise-induced hypercortisolism and decreased release of somatotropic hormones such as, for example, IGF-I (Smith L L. Cytokine hypothesis of overtraining: a physiological adaptation to excessive stress? Med Sci Sports Exerc. 2000 February; 32(2):317-31; Steinacker J M, Lormes W, Reissnecker S, Liu Y. New aspects of the hormone and cytokine response to training. Eur J Appl Physiol. 2004 April; 91(4):382-91). This circumstance results in depressed turnover of contractile proteins, decreased skeletal muscle mass, and reduced satellite cell activity in relation to replacing degenerated myofibers (Steinacker J M, Lormes W, Reissnecker S, Liu Y. New aspects of the hormone and cytokine response to training. Eur J Appl Physiol. 2004 April; 91(4):382-91). It would be therefore advantageous to prevent and/or limit the catabolically deleterious effects of TNF-α.

Recent evidence shows decreased NF-κβ activation through the oral administration of the antioxidant N-acetylcysteine (NAC) as well as through the action of the carotenoid astaxanthin on nuclear translocation of NF-κβ during inflammation and infection, following administration (Lee S J, Bai S K, Lee K S, Namkoong S, Na H J, Ha K S, Han J A, Yim S V, Chang K, Kwon Y G, Lee S K, Kim Y M. Astaxanthin inhibits nitric oxide production and inflammatory gene expression by suppressing I(kappa)B kinase-dependent NF-kappaB activation. Mol Cells. 2003 Aug. 31; 16(1):97-105; Paterson R L, Galley H F, Webster N R. The effect of N-acetylcysteine on nuclear factor-kappa B activation, interleukin-6, interleukin-8, and intercellular adhesion molecule-1 expression in patients with sepsis. Crit Care Med. 2003 November; 31(11):2574-8). This may suggest that astaxanthin and NAC, probably due to their antioxidant activity, may favor the inhibition of TNF-α-mediated catabolism in muscle cells by reducing reactive oxygen species (ROS) and/or by blocking NF-kB activation as a consequent suppression of IKK activity and IkB-α degradation (Lee S J, Bai S K, Lee K S, Namkoong S, Na H J, Ha K S, Han J A, Yim S V, Chang K, Kwon Y G, Lee S K, Kim Y M. Astaxanthin inhibits nitric oxide production and inflammatory gene expression by suppressing I(kappa)B kinase-dependent NF-kappaB activation. Mol Cells. 2003 Aug. 31; 16(1):97-105; Paterson R L, Galley H F, Webster N R. The effect of N-acetylcysteine on nuclear factor-kappa B activation, interleukin-6, interleukin-8, and intercellular adhesion molecule-1 expression in patients with sepsis. Crit Care Med. 2003 November; 31(11):2574-8).

The inhibitory action of EGCG, EGC, ECG, EC, and GCG, and/or tannic acids, singularly or in combination, complemented by the supporting action of astaxanthin and NAC, on the activation of NF-κβ-mediated signaling may reduce skeletal muscle protein breakdown in the occurrence of elevated TNF-α release as seen in response to inflammation, sepsis, infection, excessive physical stress, chronic illness, and in aging.

Without wishing to be bound by theory, it is herein believed that selective enhancement of glucose metabolism in skeletal muscle with concomitant negative modulation of glucose uptake in adipose tissue may be obtained by supplementation with EGCG, ECG, tannic acid, singularly or in combination, at bioavailable amounts. Enhanced Syntaxin 4 activity may provide increased insulin sensitivity and ameliorated glycogen accumulation in skeletal muscle, diversion of glucose utilization from lipogenic purposes, and enhanced creatine transport in muscle cells.

Creatine

The chemical structure of Creatine is as follows:

[image]

Creatine is a naturally occurring amino acid derived from the amino acids glycine, arginine, and methionine. It is readily found in meat and fish and it is also synthesized by humans. The main role of creatine is as a fuel renewal source in muscle. About 65% of creatine is stored in muscle as Phosphocreatine (creatine bound to a phosphate molecule) (Casey A, Constantin-Teodosiu D, Howell S, Hultman E, Greenhaff P L. Metabolic response of type I and II muscle fibers during repeated bouts of maximal exercise in humans. Am J Physiol. 1996 July; 271(1 Pt 1):E38-43). Muscle contractions are fueled by the dephosphorylation of adenosine triphosphate (ATP) to produce adenosine diphosphate (ADP). Without a mechanism to replenish ATP stores, ATP would be totally consumed in 1-2 seconds (Casey A, Greenhaff P L. Does dietary creatine supplementation play a role in skeletal muscle metabolism and performance? Am J Clin Nutr. 2000 August; 72(2 Suppl):607S-17S.). Phosphocreatine serves as a major source of phosphate wherein ADP is able to bind said phosphate to re-generate to form ATP which can be used in subsequent contractions. After 6 seconds of exercise, the muscle concentrations of Phosphocreatine drop by almost 50% (Gaitanos G C, Williams C, Boobis L H, Brooks S. Human muscle metabolism during intermittent maximal exercise. J Appl Physiol. 1993 August; 75(2):712-9.) as it is used to regenerate ATP. Creatine supplementation has been shown to increase the concentration of Creatine in the muscle (Harris R C, Soderlund K, Hultman E. Elevation of creatine in resting and exercised muscle of normal subjects by creatine supplementation. Clin Sci (Lond). 1992 September; 83(3):367-74.) and increase the resynthesis of Phosphocreatine within 2 minutes of recovery following exercise (Greenhaff P L, Bodin K, Soderlund K, Hultman E. Effect of oral creatine supplementation on skeletal muscle phosphocreatine resynthesis. Am J Physiol. 1994 May; 266(5 Pt 1):E725-30.).

In the early 1990's it was first clinically demonstrated that supplemental Creatine can improve strength and reduce fatigue (Greenhaff P L, Casey A, Short A H, Harris R, Soderlund K, Hultman E. Influence of oral creatine supplementation of muscle torque during repeated bouts of maximal voluntary exercise in man. Clin Sci (Lond). 1993 May; 84(5):565-71.). Resistance training with Creatine supplementation increased strength and fat-free mass over a placebo in sedentary females (Vandenberghe K, Goris M, Van Hecke P, Van Leemputte M, Vangerven L, Hespel P. Long-term creatine intake is beneficial to muscle performance during resistance training. J Appl Physiol. 1997 December; 83(6):2055-63.) as well as in male football players (Kreider R B, Ferreira M, Wilson M, Grindstaff P, Plisk S, Reinardy J, Cantler E, Almada A L. Effects of creatine supplementation on body composition, strength, and sprint performance. Med Sci Sports Exerc. 1998 January; 30(1):73-82.). In addition to increasing lean mass and strength, Creatine supplementation has been shown to increase muscle fiber cross-sectional area (Volek J S, Duncan N D, Mazzetti S A, Staron R S, Putukian M, Gomez A L, Pearson D R, Fink W J, Kraemer W J. Performance and muscle fiber adaptations to creatine supplementation and heavy resistance training. Med Sci Sports Exerc. 1999 August; 31(8):1147-56.). Moreover, high-intensity exercise performance of both males and female is improved by supplemental Creatine (Tarnopolsky M A, MacLennan D P. Creatine monohydrate supplementation enhances high-intensity exercise performance in males and females. Int J Sport Nutr Exerc Metab. 2000 December; 10(4):452-63.). It has been suggested that Creatine supplementation may also benefit individuals suffering from muscle dystrophy disorders by reducing muscle loss (Walter M C, Lochmuller H, Reilich P, Klopstock T, Huber R, Hartard M, Hennig M, Pongratz D, Muller-Felber W. Creatine monohydrate in muscular dystrophies: A double-blind, placebo-controlled clinical study. Neurology. 2000 May 9; 54(9):1848-50.). Furthermore, there is also evidence that Creatine may confer antioxidant properties (Lawler J M, Barnes W S, Wu G, Song W, Demaree S. Direct antioxidant properties of creatine. Biochem Biophys Res Commun. 2002 Jan. 11; 290(1):47-52.; Sestili P, Martinelli C, Bravi G, Piccoli G, Curci R, Battistelli M, Falcieri E, Agostini D, Gioacchini A M, Stocchi V. Creatine supplementation affords cytoprotection in oxidatively injured cultured mammalian cells via direct antioxidant activity. Free Radic Biol Med. 2006 Mar 1; 40(5):837-49.), wherein the antioxidant activity of Creatine may aid post-exercise muscle recovery.

As an additional note, Creatine retention is markedly improved with up to 60% increased efficiency through the ingestion of a concomitant carbohydrate which may be related to increased insulin concentration (Green A L, Hultman E, Macdonald I A, Sewell D A, Carbohydrate ingestion augments skeletal muscle creatine accumulation during creatine supplementation in humans. Am J Physiol. 1996 November; 271(5 Pt 1):E821-6.). Furthermore, glucose and Creatine uptake by muscle cells has been shown to be stimulated by insulin (Odoom J E, Kemp G J, Radda G K. regulation of total creatine content in a myoblast cell line. Mol Cell Biochem. 1996 May 24; 158(2):179-88.). As such, the ingestion of Creatine combined with a carbohydrate is recommended. Furthermore, it may also be beneficial to include protein at the time of Creatine ingestion (Steenge G R, Simpson E J, Greenhaff P L. Protein- and carbohydrate-induced augmentation of whole body creatine retention in humans. J Appl Physiol. 2000 September; 89(3):1165-71.).

Additionally, preliminary investigation supports a role for oral creatine supplementation in affording neuroprotection within a variety of experimental neurological disease models, including amyotrophic lateral sclerosis (ALS), Huntington's (HD) and Parkinson's (PD) diseases, as well as in the prevention of ischemic brain injury in patients at high risk of stroke (Klivenyi P, Ferrante R J, Matthews R T, Bogdanov M B, Klein A M, Andreassen O A, Mueller G, Wermer M, Kaddurah-Daouk R, Beal M F. Neuroprotective effects of creatine in a transgenic animal model of amyotrophic lateral sclerosis. Nat Med. 1999 March; 5(3):347-50; Matthews R T, Yang L, Jenkins B G, Ferrante R J, Rosen B R, Kaddurah-Daouk R, Beal M F. Neuroprotective effects of creatine and cyclocreatine in animal models of Huntington's disease. J Neurosci. 1998 Jan. 1; 18(1):156-63; Ferrante R J, Andreassen O A, Jenkins B G, Dedeoglu A, Kuemmerle S, Kubilus J K, Kaddurah-Daouk R, Hersch S M, Beal M F. Neuroprotective effects of creatine in a transgenic mouse model of Huntington's disease. J Neurosci. 2000 Jun. 15; 20(12):4389-97; Sullivan P G, Geiger J D, Mattson M P, Scheff S W. Dietary supplement creatine protects against traumatic brain injury. Ann Neurol. 2000 November; 48(5):723-9; Zhu S, Li M, Figueroa B E, Liu A, Stavrovskaya I G, Pasinelli P, Beal M F, Brown R H Jr, Kristal B S, Ferrante R J, Friedlander R M. Prophylactic creatine administration mediates neuroprotection in cerebral ischemia in mice. J Neurosci. 2004 Jun. 30; 24(26):5909-12). According to some authors, this circumstance is indicative of a close correlation between the functional capacity of the creatine kinase/phosphocreatine/creatine system and proper brain function (Wyss M, Schulze A. Health implications of creatine: can oral creatine supplementation protect against neurological and atherosclerotic disease? Neuroscience. 2002; 112(2):243-60). The animal evidence is corroborated by preliminary human studies showing the beneficial effects of oral creatine monohydrate at significantly increasing high-intensity strength in patients suffering from neuromuscular disease and mitochondrial cytopathies (Tarnopolsky M, Martin J. Creatine monohydrate increases strength in patients with neuromuscular disease. Neurology. 1999 Mar. 10; 52(4):854-7; Tarnopolsky M A, Mahoney D J, Vajsar J, Rodriguez C, Doherty T J, Roy B D, Biggar D. Creatine monohydrate enhances strength and body composition in Duchenne muscular dystrophy. Neurology. 2004 May 25; 62(10):1771-7; Tarnopolsky M A, Roy B D, MacDonald J R. A randomized, controlled trial of creatine monohydrate in patients with mitochondrial cytopathies. Muscle Nerve. 1997 December; 20(12):1502-9), and at temporarily increasing maximal isometric force in ALS patients (Mazzini L, Balzarini C, Colombo R, Mora G, Pastore I, De Ambrogio R, Caligari M. Effects of creatine supplementation on exercise performance and muscular strength in amyotrophic lateral sclerosis: preliminary results. J Neurol Sci. 2001 Oct. 15; 191(1-2):139-44). Current hypotheses of the mechanisms of creatine-mediated neuroprotection include enhanced energy storage, as well as stabilization of the mitochondrial membrane transition pore (O'Gorman E, Beutner G, Dolder M, Koretsky A P, Brdiczka D, Wallimann T. The role of creatine kinase in inhibition of mitochondrial permeability transition. FEBS Lett. 1997 Sep. 8; 414(2):253-7; Wyss M, Kaddurah-Daouk R. Creatine and creatinine metabolism. Physiol Rev. 2000 July; 80(3):1107-213). It is therefore believed that creatine improves the overall bioenergetic status of the cell, making it more resistant to injury (Zhu S, Li M, Figueroa B E, Liu A, Stavrovskaya I G, Pasinelli P, Beal M F, Brown R H Jr, Kristal B S, Ferrante R J, Friedlander R M. Prophylactic creatine administration mediates neuroprotection in cerebral ischemia in mice. J Neurosci. 2004 Jun. 30; 24(26):5909-12; Wyss M, Kaddurah-Daouk R. Creatine and creatinine metabolism. Physiol Rev. 2000 July; 80(3): 1107-213).

As used herein, a serving of the supplement comprises from about 0.1 to 10 g of creatine. A serving of the supplement, according to various embodiments comprises about 5 g of creatine per serving. In addition to, or in alternative embodiments, a serving of the supplement comprises from about 0.1 mg to about 1000 mg of Creatinol-O-phosphate. A serving of the supplement, according to embodiments one to four, as set forth in greater detail below, may comprise about 450 mg of Creatinol-O-phosphate. In a fifth embodiment, as set forth in greater detail below, a serving of the supplement may comprise about 350 mg of Creatinol-O-phosphate. Still further, in a sixth embodiment of the present invention, which is set forth in greater detail below, a serving of the supplement may comprise about 600 mg of Creatinol-O-phosphate.

Gypenosides (Phanoside)

Many chemicals derived from different plant sources have been reported to have antidiabetic properties. Gynostemma pentaphyllum, a plant that grows wild in Asia, has been used historically as an adaptogenic herb. It is traditionally used for illness-prevention and its therapeutic qualities by way of conferring antioxidant properties. One of the main active constituents of Gynostemma pentaphyllum are the dammarane-type saponins, or gypenosides.

More that 100 dammarane saponines have been characterized. Gynostemma pentaphyllum and Panax ginseng share several of these gypenosides (Megalli S, Aktan F, Davies N M, Roufogalis B D. Phytopreventative anti-hyperlipidemic effects of gynostemma pentaphyllum in rats. J Pharm Pharm Sci. 2005 Sep. 16; 8(3):507-15.). A specific gypenoside, namely phanoside, has demonstrated a potent insulin-releasing activity. Phanoside has insulin-releasing activity which is able to effect glucose metabolism (Norberg A, Hoa N K, Liepinsh E, Van Phan D, Thuan N D, Jornvall H, Sillard R, Ostenson C G. A novel insulin-releasing substance, phanoside, from the plant Gynostemma pentaphyllum. J Biol Chem. 2004 Oct. 1; 279(40):41361-7.). Furthermore, the effect of phanoside on glucose metabolism is believed to be mediated via the direct release of nitric oxide (NO) in pancreatic β-cells which, in turn, have been shown to increase glucose-induced insulin release (Norberg A, Hoa N K, Liepinsh E, Van Phan D, Thuan N D, Jornvall H, Sillard R, Ostenson C G. A novel insulin-releasing substance, phanoside, from the plant Gynostemma pentaphyllum. J Biol Chem. 2004 Oct. 1; 279(40):41361-7; Tanner M A, Bu X, Steimle J A, Myers P R. The direct release of nitric oxide by gypenosides derived from the herb Gynostemma pentaphyllum. Nitric Oxide. 1999 October; 3(5):359-65; Nakata M, Yada T. Endocrinology: nitric oxide-mediated insulin secretion in response to citrulline in islet beta-cells. Pancreas. 2003 October; 27(3):209-13.).

As used herein, a serving of the supplement comprises from about 0.1 mg to 1,200 mg of Gynostemma pentaphyllum comprising Gypenosides and/or Phanoside or derivatives thereof. A serving of the supplement, according to embodiments one to four, as set forth in greater detail below, may comprise about 500 mg of Gypenosides and/or Phanosides. In a fifth embodiment, as set forth in greater detail below, a serving of the supplement may comprise about 700 mg of Gypenosides and/or Phanosides. Still further, in a sixth embodiment of the present invention, which is set forth in greater detail below, a serving of the supplement may comprise about 1,000 mg of Gypenosides and/or Phanosides.

N-acetyl Cysteine

N-acetyl cysteine (NAC), a naturally-occurring derivative of the amino acid cysteine, is produced in the body. It is found in many foods and is also an intermediary in the conversion of cysteine to glutathione. Furthermore, NAC is thought to benefit the immune system as an antioxidant. The conversion product of NAC, glutathione, is the body's primary antioxidant which is extremely important to immune functions (Droge W, Breitkreutz R. Glutathione and immune function. Proc Nutr Soc. 2000 November; 59(4):595-600). Moreover, it has been shown that NAC is capable of replenishing depleted glutathione levels associated with HIV infection (De Rosa S C, Zaretsky M D, Dubs J G, Roederer M, Anderson M, Green A, Mitra D, Watanabe N, Nakamura H, Tjioe I, Deresinski S C, Moore W A, Ela S W, Parks D, Herzenberg L A, Herzenberg L A. N-acetylcysteine replenishes glutathione in HIV infection. Eur J Clin Invest. 2000 October; 30(10):915-29).

As used herein, a serving of the supplement comprises from about 0.1 mg to 1,000 mg of N-acetyl cysteine. A serving of the supplement, according to embodiments one to five, as set forth in greater detail below, may comprise about 500 mg of N-acetyl cysteine. In a sixth embodiment, as set forth in greater detail below, a serving of the supplement may comprise about 600 mg of N-acetyl cysteine.

Epigallocatechin Gallate

Epigallocatechin gallate (ECGC), which makes up 10-50% of the total catechins, is a member of the active Catechin polyphenol family of Green Tea, also comprising Epicatechin Gallate (ECG) and Tannic Acid. (Kao Y H, Hiipakka R A, Liao S. Modulation of endocrine systems and food intake by green tea epigallocatechin gallate. Endocrinology. 2000 March; 141(3):980-7). EGCG displays potent antioxidant activity as shown by laboratory tests. It has been shown to be greater than many other well-established antioxidants such as vitamin C and vitamin E (Pillai S P, Mitscher L A, Menon S R, Pillai C A, Shankel D M. Antimutagenic/antioxidant activity of green tea components and related compounds. J Environ Pathol Toxicol Oncol. 1999; 18(3):147-58). Moreover, in humans, administration of Green Tea extracts rich in EGCG and other catechins have been shown induce a rapid increase in plasma antioxidant activity (Benzie I F, Szeto Y T, Strain J J, Tomlinson B. Consumption of green tea causes rapid increase in plasma antioxidant power in humans. Nutr Cancer. 1999; 34(1):83-7) and aid in weight loss due to increased metabolism and fat oxidation (Chantre P, Lairon D. Recent findings of green tea extract AR25 (Exolise) and its activity for the treatment of obesity. Phytomedicine. 2002 January; 9(1):3-8; Dulloo A G, Duret C, Rohrer D, Girardier L, Mensi N, Fathi M, Chantre P, Vandermander J. Efficacy of a green tea extract rich in catechin polyphenols and caffeine in increasing 24-h energy expenditure and fat oxidation in humans. Am J Clin Nutr. 1999 December; 70(6): 1040-5).

As used herein, a serving of the dietary supplement comprises a source of EGCG, ECG, and/or Tannic Acid, wherein the supplement comprise from about 0.1 mg to about 1,000 mg for each of said EGCG, ECG, and Tannic Acid individually. In combination, according to various embodiments of the present invention, the total EGCG, ECG, and Tannic acid content of a serving comprises from about 0.1 mg to about 1,600 mg. A serving of the supplement, according to embodiments one to four, as set forth in greater detail below, may comprise about 250 mg of EGCG. In the fifth and sixth embodiments, as set forth in greater detail below, a serving of the supplement may comprise about 350 mg of EGCG.

Astaxanthin

Astaxanthin is a red carontenoid pigment occurring naturally in many living organisms. Studies utilizing animals indicate that astaxanthin has antioxidant activity that can attenuate exercise-induced muscle damage (Aoi W, Naito Y, Sakuma K, Kuchide M, Tokuda H, Maoka T, Toyokuni S, Oka S, Yasuhara M, Yoshikawa T. Astaxanthin limits exercise-induced skeletal and cardiac muscle damage in mice. Antioxid Redox Signal. 2003 February; 5(1):139-44), has anticancer activity (Jyonouchi H, Sun S, lijima K, Gross M D. Antitumor activity of astaxanthin and its mode of action. Nutr Cancer. 2000; 36(1):59-65), anti-inflammatory activity (Kurashige M, Okimasu E, Inoue M, Utsumi K. Inhibition of oxidative injury of biological membranes by astaxanthin. Physiol Chem Phys Med NMR. 1990; 22(1):27-38), anti-diabetic activity (Uchiyama K, Naito Y, Hasegawa G, Nakamura N, Takahashi J, Yoshikawa T. Astaxanthin protects beta-cells against glucose toxicity in diabetic db/db mice. Redox Rep. 2002; 7(5):290-3), immunity-boosting properties (Okai Y, Higashi-Okai K. Possible immunomodulating activities of carotenoids in in vitro cell culture experiments. Int J Immunopharmacol. 1996 December; 18(12):753-8), and antihypertensive and neuroprotective properties (Hussein G, Nakamura M, Zhao Q, Iguchi T, Goto H, Sankawa U, Watanabe H. Antihypertensive and neuroprotective effects of astaxanthin in experimental animals. Biol Pharm Bull. 2005 January; 28(1):47-52).

As used herein, a serving of the supplement comprises about 1 mg to about 20 mg of astaxanthin. A serving of the supplement, according to embodiments one to four, as set forth in greater detail below, may comprise about 7.5 mg of astaxanthin. In the fifth and sixth embodiments, as set forth in greater detail below, a serving of the supplement may comprise about 15 mg of astaxanthin.

Additionally, various embodiments of the present may comprise a protein, or a source of protein. Various embodiments may also comprise amino acids, such as, but limited not to, Leucine, Isoleucine, Valine, Histidine, Lysine, Methionine, Phenylalanine, Threonine and Tryptophan, as set forth in greater detail in the examples in this disclosure.

Furthermore, various embodiments of the present may comprise a carbohydrate, or a source of carbohydrate. Still further, various embodiments of the present invention may comprise a sugar or a source of sugars. Various embodiments may comprise sugars, such as, but not limited to, Dextrose, Fructose, and Maltodextrin, as set forth in greater detail in the examples in this disclosure.

The additional energy and nutrients provided by the dietary supplement may avoid interfering with or diminishing the physiological anabolic response to protein sources and other nutrients consumed as part of regular daily meals. Due to its modest caloric density, the dietary supplement is suitable to be consumed with calorie-reduced-dietary-regimens, and is appropriate for individuals suffering from a reduced appetite, such as, for example, the ill and the elderly, for whom consumption of energetically-rich food supplements often blunts the stimulus to ingest nutritiously complete regular meals. Various embodiments of the present invention may be beneficial to professional and recreational athletes, as well as active individuals, patients recovering from injury or illness, the elderly, and persons suffering from wasting syndromes.

Repeated consumption of the disclosed dietary supplement according to the described methods may be a beneficial nutritional support for the prevention of skeletal muscle catabolism as induced by lack of specific nutrients, excessive exertion, overtraining and/or stress, prevention and treatment of muscle atrophy and muscle protein wasting due to disuse, such as in the case of injury, immobilization and/or bed rest confinement, and ageing and/or age-related loss of muscle mass and strength. Additionally, given the enhanced creatine transport activity in myocytes and neurons, the ameliorated glucose metabolism in muscle fibers, and the improved skeletal muscle work capacity, it is believed that repeated consumption of the dietary supplement may provide an effective prophylactic and therapeutic aid against such neurodegenerative conditions as Amyotrophic Lateral Sclerosis, Huntington's Disease and Parkinson's Disease, as well as in the minimization of ischemic brain injury in patients at high risk of stroke. In such occurrences, the dietary supplement may help preserve residual muscle contractility and the integrity of neuromuscular functions.

The dietary supplement, according to various embodiments may comprise one or more of high to moderate-glycemic index carbohydrates, dammarane saponins from Gynostemma pentaphyllum, ester-bond containing polyphenols, creatine, and related guanidine compounds. According to the various embodiments of the present invention, the composition may take the form of a dietary supplement which may be consumed in any form. For example, the dosage form of the supplemental dietary supplement may be provided as, e.g., a powder beverage mix, a liquid beverage, a ready-to-eat bar or drink product, a capsule, a tablet, a caplet, or as a dietary gel. The most preferred dosage form is powdered beverage mixture.

Furthermore, the dosage form of the dietary supplement, in accordance with any embodiment of the present invention, may be provided in accordance with customary processing techniques for herbal and/or dietary supplements in any of the forms mentioned above. Those of skill in the art will appreciate that the dietary supplement may contain a variety of, and any number of different, excipients.

### EXAMPLES

### Example 1

A serving of the dietary supplement comprises the following ingredients in powdered beverage mix form. The dietary supplement may, for example, be mixed in 360 ml-450 ml water. This example may be particularly suitable for sports uses. The dietary supplement comprises for example: Dextrose (25 g), Fructose (10 g), Leucine (1.59 g), Isoleucine (0.85 g), Valine (1 g), Histidine (0.92 g), Lysine (1.32 g), Methionine (0.27 g), Phenlyalanine (1.32 g), Threonine (1.25 g), Creatine monohydrate (5 g), Gypenosides/Phanoside (500 mg), N-acetyl cysteine (500 mg), Creatinol-O-phosphate (450 mg), EGCG (250 mg), and Astaxanthin (7.5 mg).

### Example 2

A serving of the dietary supplement comprises the following ingredients in powdered beverage mix form. The dietary supplement may, for example, be mixed in 360 ml-450 ml water. This example may also be particularly suitable for sports uses. The dietary supplement comprises for example: Dextrose (14 g), Maltodextrin (14 g), Leucine (3.7 g), Isoleucine (1.98 g), Valine (2.31 g), Creatine monohydrate (5 g), Gypenosides/Phanoside (500 mg), N-acetyl cysteine (500 mg), Creatinol-O-phosphate (450 mg), EGCG (250 mg), and Astaxanthin (7.5 mg).

### Example 3

A serving of the dietary supplement comprises the following ingredients in powdered beverage mix form. The dietary supplement may, for example, be mixed in 360 ml-450 ml water. This example may also be particularly suitable for sports uses. The dietary supplement comprises for example: Dextrose (14 g), Maltodextrin (14 g), Leucine (3.5 g-8 g), Creatine monohydrate (5 g), Gypenosides/Phanoside (500 mg), N-acetyl cysteine (500 mg), Creatinol-O-phosphate (450 mg), EGCG (250 mg), and Astaxanthin (7.5 mg).

### Example 4

A serving of the dietary supplement comprises the following ingredients in powdered beverage mix form. The dietary supplement may, for example, be mixed in 360 ml-450 ml water. This example may also be particularly suitable for sports uses. The dietary supplement comprises for example: Dextrose (30 g), Fructose (10 g), Creatine monohydrate (5 g), Gypenosides/Phanoside (500 mg), N-acetyl cysteine (500 mg), Creatinol-O-phosphate (450 mg), EGCG (250 mg), and Astaxanthin (7.5 mg).

### Example 5

A serving of the dietary supplement comprises the following ingredients in powdered beverage mix form. The dietary supplement may, for example, be mixed in 360 ml-450 ml water. This example may be particularly suitable for elderly individuals and chronically ill patients. This example may be consumed 3 times/day. The dietary supplement comprises for example: Dextrose (15 g), Fructose (15 g), Leucine (3.2 g), Isoleucine (1 g), Valine (2.1 g), Lysine (2.6 g), Histidine (1.7 g), Methionine (0.5 g), Phenlyalanine (2.2 g), Threonine (2.1 g), Tryptophan (0.6 g), Creatine monohydrate (5 g), Gypenosides/Phanoside (700 mg), N-acetyl cysteine (500 mg), Creatinol-O-phosphate (350 mg), EGCG (350 mg), and Astaxanthin (15 mg).

### Example 6

A serving of the dietary supplement comprises the following ingredients in powdered beverage mix form. The dietary supplement may, for example, be mixed in 360 ml-450 ml water. This example may also be particularly suitable for neuroprotection. This example may be consumed 3 times/day. The dietary supplement comprises for example: Dextrose (25 g), Fructose (10 g), Leucine (3.2 g), Isoleucine (1 g), Valine (2.1 g), Creatine monohydrate (5 g), Gypenosides/Phanoside (1 g), N-acetyl cysteine (600 mg), Creatinol-O-phosphate (600 mg), EGCG (350 mg), and Astaxanthin (15 mg).

What is claimed:

1. A dietary supplement comprising:
a source of at least one of epigallocatechin gallate (EGCG), epicatechin gallate (ECG), epicatechin (EC), tannic acid or related catechins; and
a source of Gypenosides.

2. The dietary supplement of claim 1, further comprising a source of N-acetyl cysteine.

3. The dietary supplement of claim 2, further comprising a source of Astaxanthin.

4. The dietary supplement of claim 3, further comprising a source of Carbohydrates.

5. The dietary supplement of claim 4, further comprising a source of Proteins or Amino acids or derivatives thereof.

6. The dietary supplement of claim 5, further comprising a source of Creatine or derivatives thereof.

7. The dietary supplement of claim 6, further comprising Creatinol-O-phosphate.

8. The dietary supplement of claim 1, further comprising a source of Astaxanthin.

9. The dietary supplement of claim 1, further comprising a source of Carbohydrates.

10. The dietary supplement of claim 1, further comprising a source of Proteins or Amino acids or derivatives thereof.

11. The dietary supplement of claim 1, further comprising a source of Creatine or derivatives thereof.

12. The dietary supplement of claim 1, further comprising Creatinol-O-phosphate.

13. A dietary supplement comprising:
from about 250 mg to about 350 mg of at least one of epigallocatechin gallate (EGCG), epicatechin gallate (ECG), epicatechin (EC), tannic acid or related catechins;
from about 500 mg to about 1 g of Gypenosides;
from about 500 mg to about 600 mg of N-acetyl cysteine;
from about 7.5 mg to about 15 mg of Astaxanthin;
from about 28 g to about 40 g of Carbohydrate;
from about 3.5 g to about 16 g of Proteins or Amino acids or derivatives thereof;
about 5 g of Creatine or derivatives therefore;
from about 450 mg to about 600 mg of Creatinol-O-phosphate.

14. A method of decreasing muscle catabolism and increasing muscle size and strength in a human or animal, comprising the step of:
administering a dietary supplement comprising a source of at least one of epigallocatechin gallate (EGCG), epicatechin gallate (ECG), epicatechin (EC), tannic acid or related catechins and further comprising a source of Gypenosides.

15. The method of claim 14, wherein the dietary supplement further comprises a source of N-acetyl cysteine.

16. The method of claim 15, wherein the dietary supplement further comprises a source of Astaxanthin.

17. The method of claim 16, wherein the dietary supplement further comprises a source of Carbohydrates.

18. The method of claim 17, wherein the dietary supplement further comprises a source of Proteins or Amino acids or derivatives thereof.

19. The method of claim 18, wherein the dietary supplement further comprises a source of Creatine or derivatives thereof.

20. The method of claim 19, wherein the dietary supplement further comprises Creatinol-O-phosphate.

21. A method for enhancing GLUT4 protein translocation to the plasma membrane in non-adipose cells in a human or animal, comprising the step of:
administering a dietary supplement comprising a source of at least one of epigallocatechin gallate (EGCG), epicatechin gallate (ECG), epicatechin (EC), tannic acid or related catechins; and a source of Gypenosides.

22. The method of claim 21, wherein the dietary supplement further comprises a source of N-acetyl cysteine.

23. The method of claim 22, wherein the dietary supplement further comprises a source of Astaxanthin.

24. The method of claim 23, wherein the dietary supplement further comprises a source of Carbohydrates.

25. The method of claim 24, wherein the dietary supplement further comprises a source of Proteins or Amino acids or derivatives thereof.

26. The method of claim 25, wherein the dietary supplement further comprises a source of Creatine or derivatives thereof.

27. The method of claim 26, wherein the dietary supplement further comprises Creatinol-O-phosphate.
